(19) United States
(12) Patent Application Publication
(10) Pub. No.: US 2012/0083498 A1
(43) Pub. Date: Apr. 5, 2012

(54) Modulators of Viral Transcription, and Methods and Compositions Therewith

(76) Inventor: Fatah Kashanchi, Manassas, VA (US)

(21) Appl. No.: 13/162,832

(22) Filed: Jun. 17, 2011

Related U.S. Application Data
(60) Provisional application No. 61/355,711, filed on Jun. 17, 2010.

(51) Int. Cl. A61K 31/53; A61P 31/18

(52) U.S. Cl. 514/245

(57) ABSTRACT

Processes to treat human immunodeficiency virus (HIV) infection are included.

### CROSS-REFERENCE TO RELATED APPLICATIONS

This application claims the benefit of U.S. Provisional Application No. 61/355,711, filed 17 Jun. 2010, entitled “A Derivative of Roscovitine as an Effective Inhibitor of HIV-1 Transcription,” which is hereby incorporated by reference in its entirety.

### STATEMENT REGARDING FEDERALLY SPONSORED RESEARCH OR DEVELOPMENT

This invention was made with government support under Grant Number 5R21AI065236-02, 1R21AI065236-01A1 awarded by the National Institute of Health (NIH). The government has certain rights in the invention.

### BRIEF DESCRIPTION OF THE SEVERAL VIEWS OF THE DRAWINGS

FIG. 1 shows TABLE 1 showing the screening results of various CDK inhibitors in HIV-1 cell killing assay. Results of this screen show 19 inhibitors where percent of dead cells are indicated after various drug treatments. A number of drugs caused death in HIV-1 infected cells much more efficiently than uninfected cells. The inhibitors were classified into three categories: high, moderate or poor, according to their effect on cellular viability in both HIV-1 infected and uninfected cells.

FIG. 2 shows TABLE 2 showing the screening results of Roscovitine/CR8 derivatives in HIV-1 cell killing assay. Results of such a screen show the 18 CR8 derivatives where percent of dead cells are indicated after various drug treatments. The inhibitors were classified into three categories: high, moderate or poor, according to their effect on cellular viability in both HIV-1 infected and uninfected cells. Interestingly, the two derivatives that caused maximum death in infected cells were minimally toxic to uninfected control cells.

FIGS. 3-4 show screening results of CR8 derivatives on Tat-dependent transcription HIV-1 LTR. A. is a bar graph demonstrating raw luciferase units with 18 CR8 derivatives. TZM-b1 cells were transfected with lug of Tat and treated the next day with DMSO, or the indicated CR8 derivative compounds at 50 nM. 48 hrs-post drug treatment, luciferase activity of the firefly luciferase was measured with the BrightGlo Luciferase Assay and luminescence was read from a 96 well plate on an EG&G Berthold luminometer. Assays were performed in triplicate, average and standard deviations are shown. B. shows a bar graph demonstrating percent viability based on MTT assays with two different doses (50 nM and 10 μM) of DMSO, CR8, MRT-033, and BJFP1154 (CR8#13) tested on infected ACH2, J1.1, OM10.1, U1 and uninfected CEM, Jurkat T cells, and U937 cells.

FIGS. 5-7 (panels A-D) shows western blots demonstrating the effect of drugs on Tat-mediated transactivation in HCT116 WT and HCT116 Dicer-/- cells. pHIV-1 LTR-CAT (1μg) construct was transfected in 2×106 cells in the absence or presence of Tat (1 μg). Six hours later, the transfected cells were treated with DMSO, Flavopirodol (100 nM), CR8#13 (100 nM), F07#13 (100 nM), and 9AA (1000 nM). Treated cells were incubated in complete DMEM for 48 hrs at 37° C. Cells were harvested and cell extracts were used for CAT analysis. One tenth the amount of HCT116 Dicer-/- extract compared to HCT116 WT was used for CAT analysis. A. is a western blot that shows results from HCT116 WT cells and panel and B. is a western blot that shows results from HCT116 Dicer-/- cells. The corresponding bar graphs in A and B show values that represent the percentage of conversion of the [14C] chloramphenicol substrate in the CAT assay. C. is a western blot for Dicer and PIWIL4 in the HCT116 WT and Dicer -/- cells. One hundred micrograms of total extracts were run on a 4-20% (w/v) SDS/PAGE and western blotted for presence of Dicer, PIWIL4 and actin. D. is a western blot from a CAT assay of Dicer WT HCT116 cells that had been treated with siRNA against Dicer, transfection with Tat followed by drug treatment.

FIGS. 8-10 (panels A-C) is a panel of western blots and a bar graph. A. is a western blot (50 μg of total protein) for Dicer, Drosha, Ago2 and PIWIL4 in control T- cells (CEM) and monocytes (U937). Dicer protein expression becomes apparent only after PMA treatment resulting in differentiation of cells into macrophages. PIWIL4 are present in both cell types. B. is a western blot of monocytes (U937) and monocytes (U937) treated with PMA. C. is a graphical representation demonstrating results from reverse transcriptase (RT) reaction assays to determine virus production in drug treated cells. Jurkat T-cell and promonocytic U937 cells were electroporated with 5 μg pNL4.3 followed the next day by drug treatment of Flavopiridol (200 nM), CR8 (100 nM) or CR8#13 (50 nM). Cell supernatants were collected at 48 hours post drug treatment. Viral supernatants (10 μl ) were incubated in a 96-well plate with reverse transcriptase (RT) reaction mixture overnight at 37° C., and 10 μl of the reaction mix was spotted on a DEAE Filtermat paper, washed with 5% (w/v) Na2HPO4 followed by water wash, and then dried completely. RT activity was measured in a Betaplate counter.

FIG. 11 shows bands from agarose gels demonstrating a lack of effect on cellular genes controlled by cdk9 after treatment with CR8#13. 293T cells were treated with three different concentrations of CR8#13 (20 nM, 50 nM, and 200 nM). Cells were processed 48hrs-post treatment for RT-PCR. Effector cdk9 genes such as CIITA, IL-8, CAD, MCL-1, Cyclin D1, and PBX-1 were used in the RT-PCR.

FIGS. 12-16 (panels A-E) A. is a model of the effect of RNA polymerase II phosphorylation on transcription. RNA polymerase II CTD is hypo-phosphorylated at the initiation complex; Ser5 is only phosphorylated at the promoter clearance stage; and Ser2 is mostly phosphorylated at the elongation phase. HIV-1 genome is unique in that it contains both Ser2 and Ser5 phosphorylation at the elongation stage (Zhou et al, 2004). Phosphorylation of Ser2 and Ser5 could be seen by multiple cyclin/cdk complexes. B. are bands from a gel demonstrating small RNA fragments corresponding to TAR sequence from RNase protection assays. Ten micrograms of total RNA from TNF treated CEM (lane 1) and TNF treated ACH2 cells (lanes 2-6) were hybridized to a radiolabeled TAR 5′ probe and then treated with RNase A. Arrows indicate the probe protected by TAR at 27 nucleotides and the probe protected by a TAR miRNA at approximately 22 nt. Cyc202 concentration at 500 nM, CR8 at 100 nM, CR8#13 at 50 nM, and Flavopiridol at 50 nM were used for these experiments. C. are bands from a gel demonstrating results from ACH2 cells that were treated with Flavopiridol (50 nM), CR8 (100 nM) and CR8#13 (100 nM). RNA was extracted 48 hrs-post drug treatment. 500 ng of RNA from the microRNA-enriched fraction was used to generate cDNA using the Quantimir kit (SBI). RT reactions are performed followed by PCR in which a universal reverse primer is provided by the manufacturer. Specific microRNA forward primers are identical in sequence to the microRNA of interest. PCR products corresponding to the amplified microRNAs were resolved in a 3.5% (w/v) agarose gel. The PCR products are at around 67 bp as compared with the Fermentas 1 kb DNA Plus Ladder. Increased amounts of 3′ and 5′ TAR microRNA were observed post drug treatment. D. are bands from a gel demonstrating results from total RNA (lug) from each samples was separated in a 1% (w/v) agarose gel. The location of both 18S and 28S are shown. E. is a bar graph demonstrating results from a RT assay that was performed to detect viral levels in ACH2 cells after TNF and drug treatments. ACH2 cells were treated with Flavopiridol (50 nM), Cyc202 (500 nM), CR8 (100 nM) and CR8#13(100 nM). Supernatants were collected 48 hrs later and used for RT assay. TNF treatment significantly increased RT levels in ACH2 cells and drug treatment was able to decrease RT levels.

FIGS. 17-22 (panels A-F) A. is a model of TZM-b1 cells suppression and activation. Trichostatin-A (TSA), a widely used HDAC inhibitor were used to activate the integrated HIV-1 LTRLuc transcription in TZM-b1 and abolish the repressive heterochromatic state. Seven days post treatment of TSA, the TZM-b1 were transfected with the TAR microRNA. B. are bands from a gel demonstrating chromatin changes using antibodies specific for inhibitory factors verified by ChIP assays performed with the TSA treated TZM-b1s. Primers specific for the HIV-1 LTR were used to amplify DNA that was precipitated with each antibody. MicroRNA machinery (Ago2), histone methyltransferases (Suv39H1), chromatin remodeling markers (SETDB1, SETMAR), and transcription repressors (PIASγ) were downregulated after TSA treatment on the integrated HIV-LTR. C. are bands from a gel demonstrating results from ChIP assays that were performed on several markers of chromatin repression (HDAC1) and microRNA machinery (Ago2) in TZMb1 cells. Primers specific for the HIV-1 LTR were used to amplify DNA that was precipitated with each antibody. Lane 1 shows basal levels of repressive markers on the HIV-1 LTR. Lane 2 shows that seven days of TSA treatment removes the markers of repressive chromatin. Lane 3 shows that the TAR-D mutant does not initiate a recruitment of repressive enzymes. Lane 4 demonstrates that addition of the WT-TAR molecule is sufficient to recruit Ago2 and HDAC1 back to the HIV-1 LTR region. D. are bands from a gel demonstrating results from TZM-b1 cells that were treated with Flavopiridol (50 nM), CR8 (100 nM) and CR8#13 (100 nM) after 7-day TSA treatment. RNA was extracted 48hrs-post drug treatment. 500 ng of RNA from the microRNA-enriched fraction was used to generate cDNA using the Quantimir kit (SBI) in order to poly adenylate small RNA species. RT reactions were performed followed by PCR in which a universal reverse primer was provided by the manufacturer. Specific microRNA forward primers are identical in sequence to the microRNA of interest. PCR products corresponding to the amplified microRNAs were separated in a 3.5% (w/v) agarose gel. The PCR products are at around 67 bp as compared with the Fermentas 1 kb DNA Plus Ladder. Increased levels of 3′TAR microRNA were produced post CR8#13 treatment. E. are bands from a gel demonstrating results from ChIP assays that were performed on several markers of chromatin repression (HDAC1, Suv39H1) and microRNA machinery (Ago2) in TZMb1 cells. Primers specific for the HIV-1 LTR were used to amplify DNA that was precipitated with each antibody. Lane 1 indicates basal levels of repressive markers on the HIV-1 LTR. Lane 2 indicates that seven days of TSA treatment removes the markers of repressive chromatin and Lane 3 shows results that the CR8#13 treatment is sufficient to recruit HDAC1, Ago2 and Suv39H1 back to the HIV-1 LTR region. F. is a bar graph demonstrating results from luciferase assays that were performed on the cells used in FIG. 6E. Luciferase activity increased with TSA treatment and then decreased post-CR8#13 treatment.

FIG. 23 depicts a non-limiting non-binding model for cdk inhibitor-mediated viral microRNA production and transcriptional inhibition. During viral transcription, Tat/pTEF-b complexes increase phosphorylation of RNA polymerase II, leading to increased transcriptional elongation. In contrast, cdk inhibitors reduce phosphorylation of RNA polymerase II (at either Ser 2, 5 or both), consequently decreasing elongation. As a result, increased TAR transcripts are produced which aid in the recruitment of RNA interference machinery and heterochromatin remodeling complexes to the HIV-1 promoter, inhibiting transcription. This form of inhibition may ultimately lead to DNA methylation as a permanent epigenetic mark on HIV-1 LTR.

FIGS. 24-27 depict chemical structures of CR8#13(BJFP1154), MRT3-033, MRT3-028, MRT3-012, MRT3-038, MRT3-041, MRT3-039, MRT3-024, and MRT3-040.

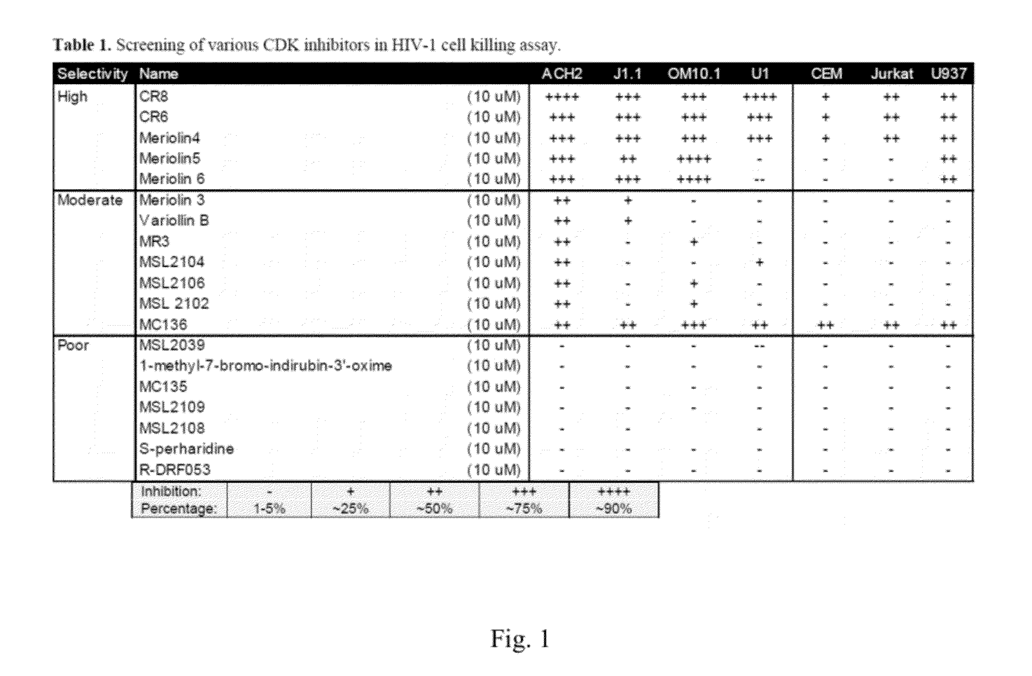

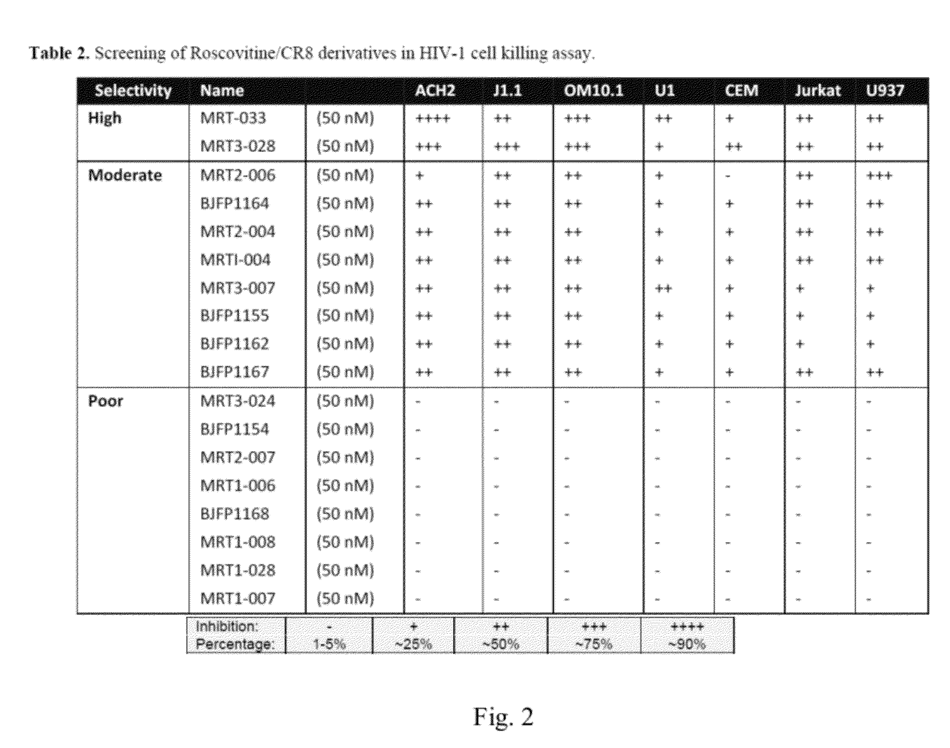

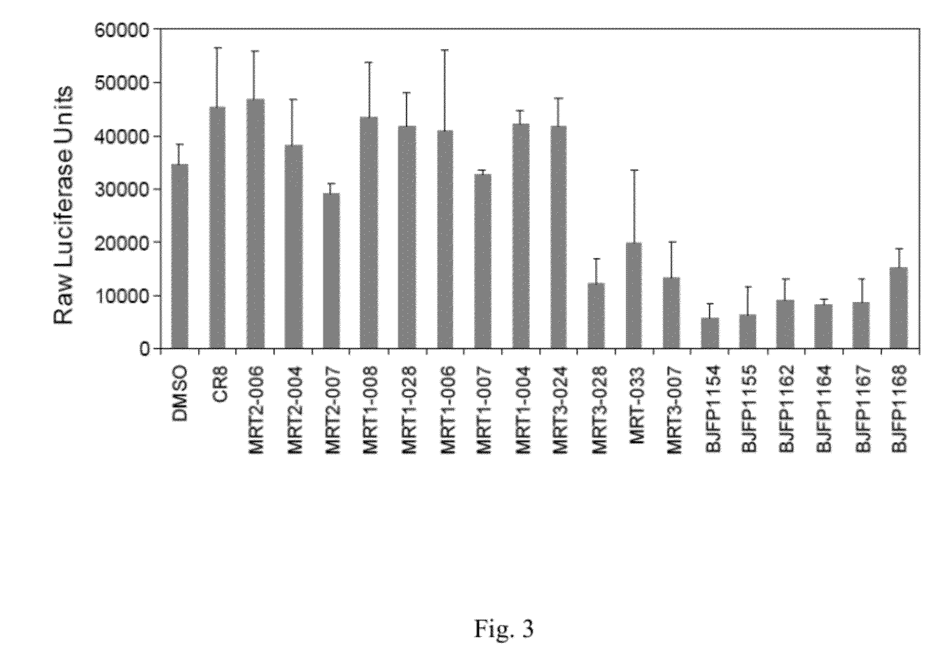

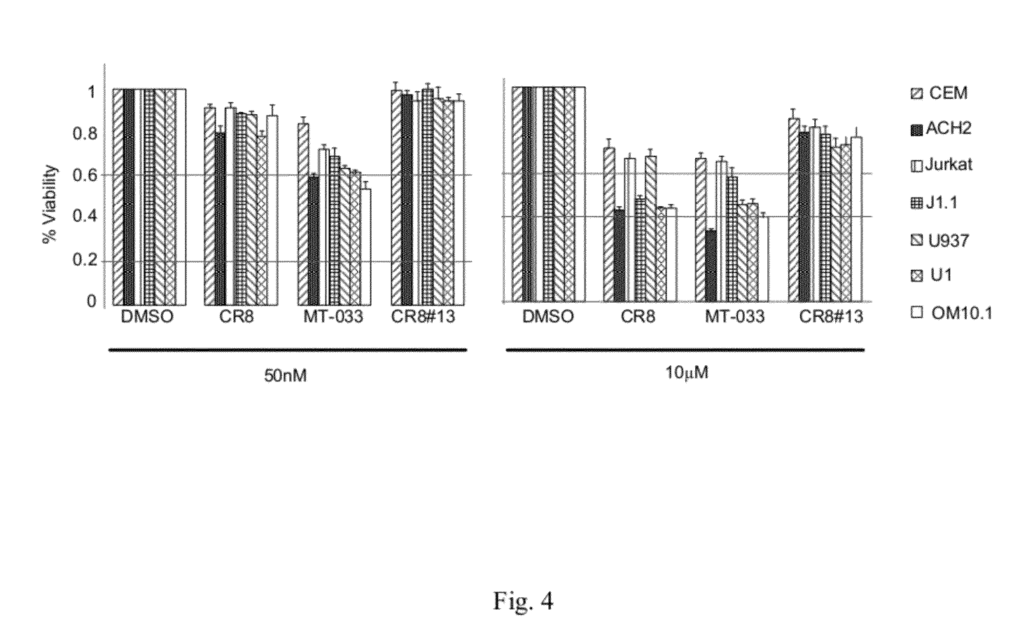

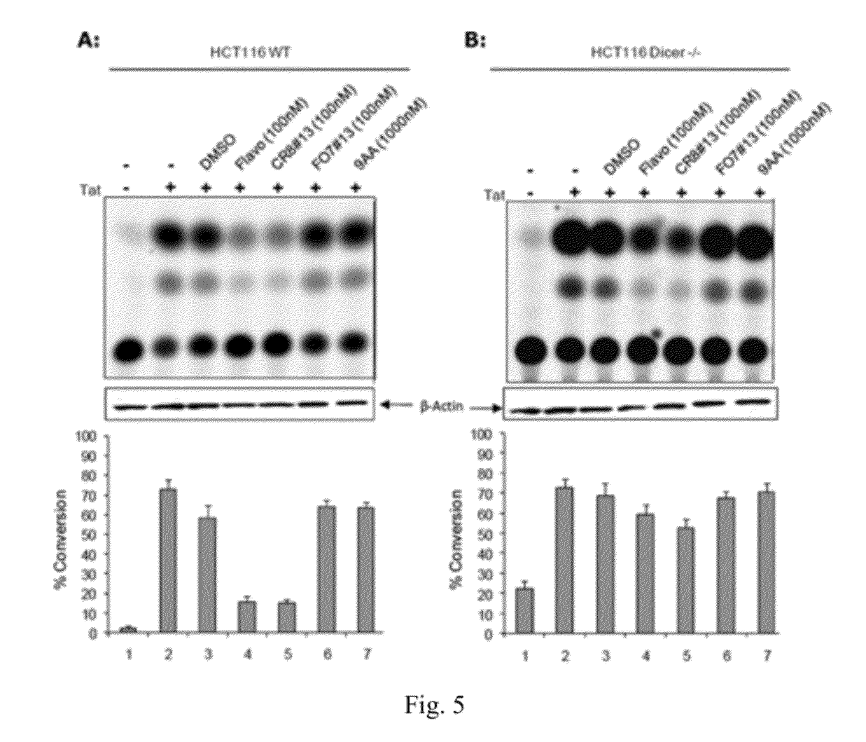

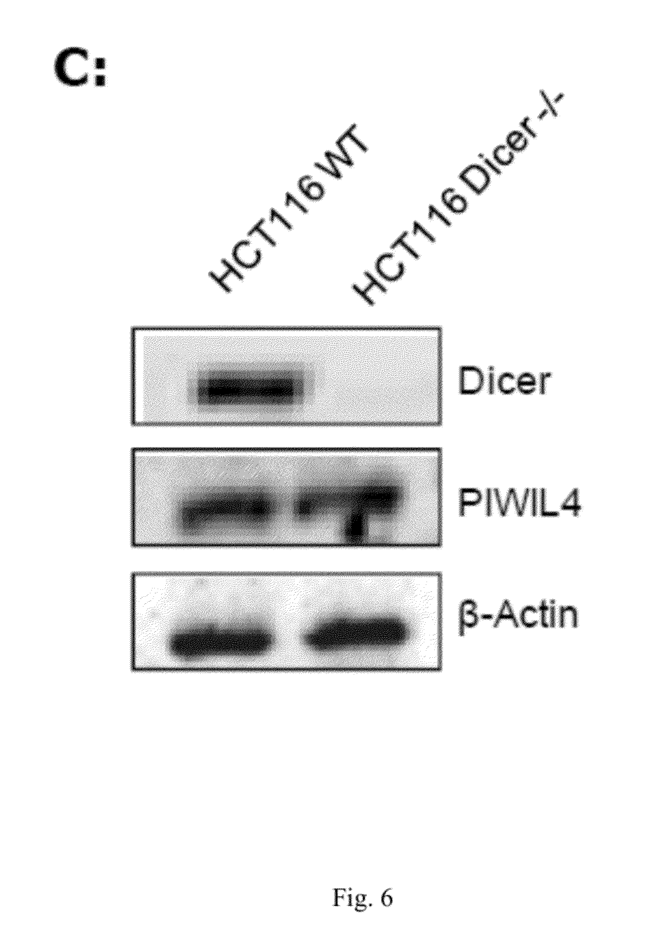

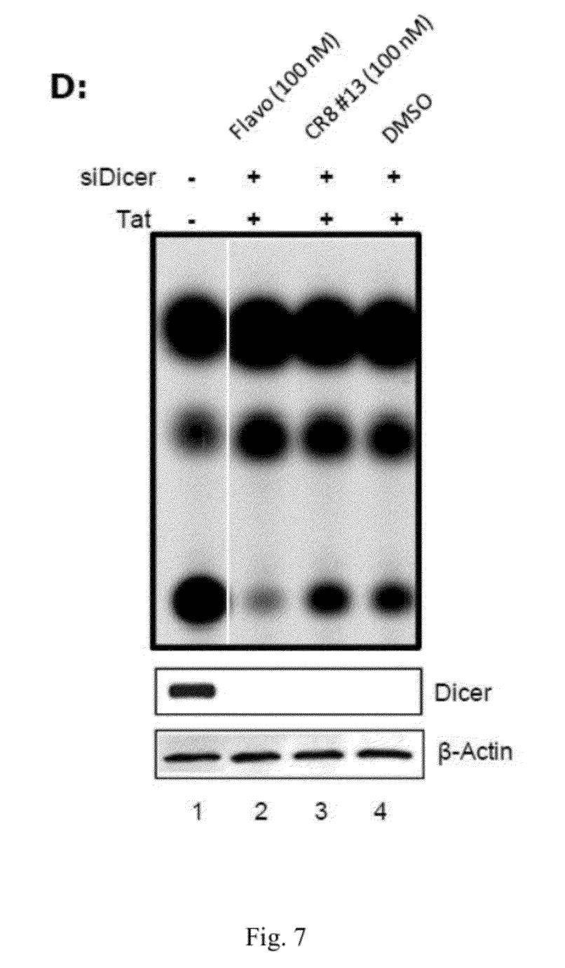

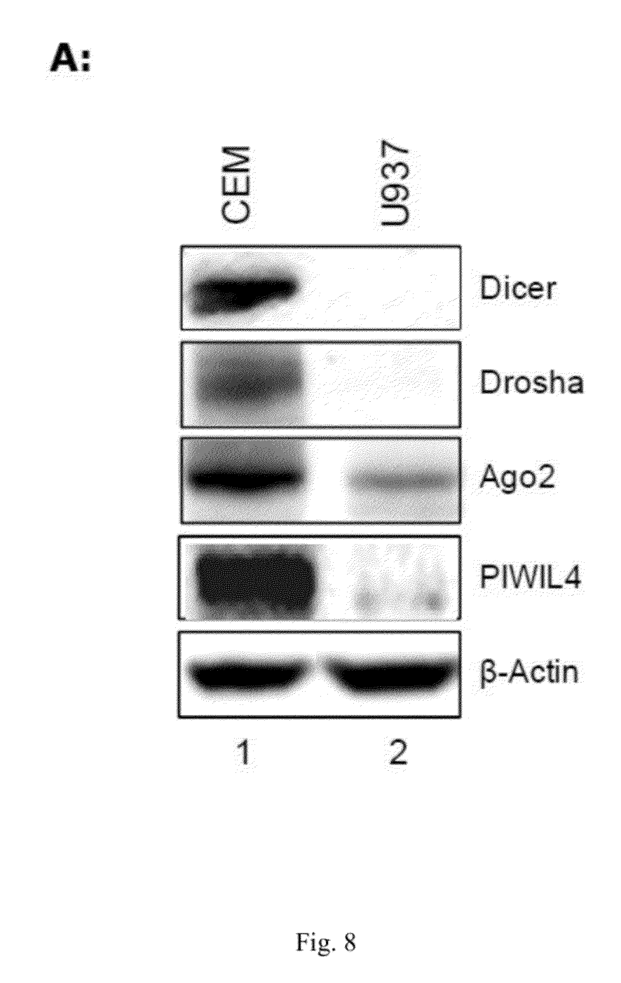

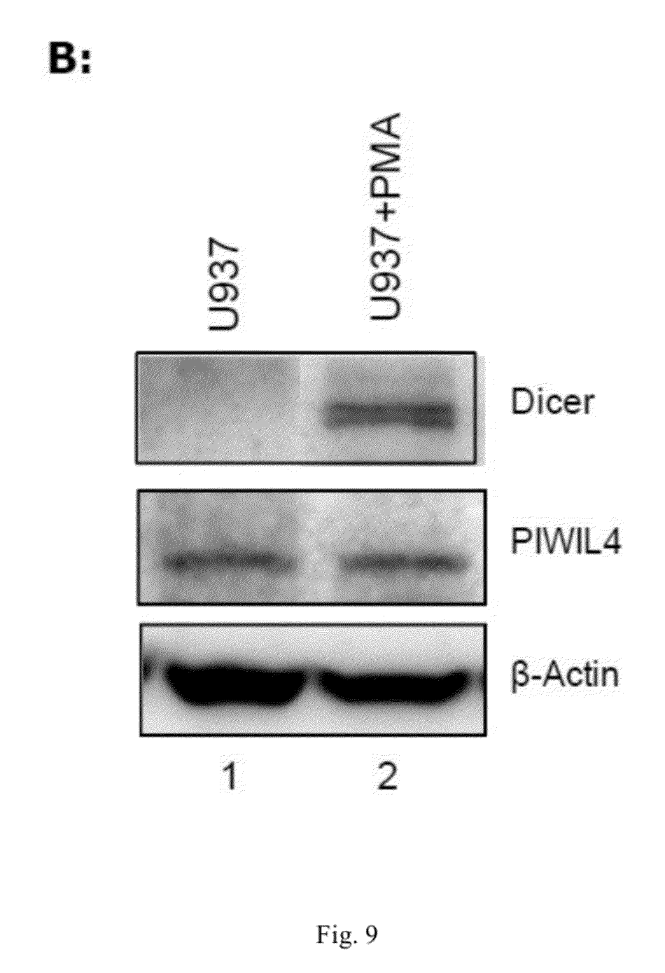

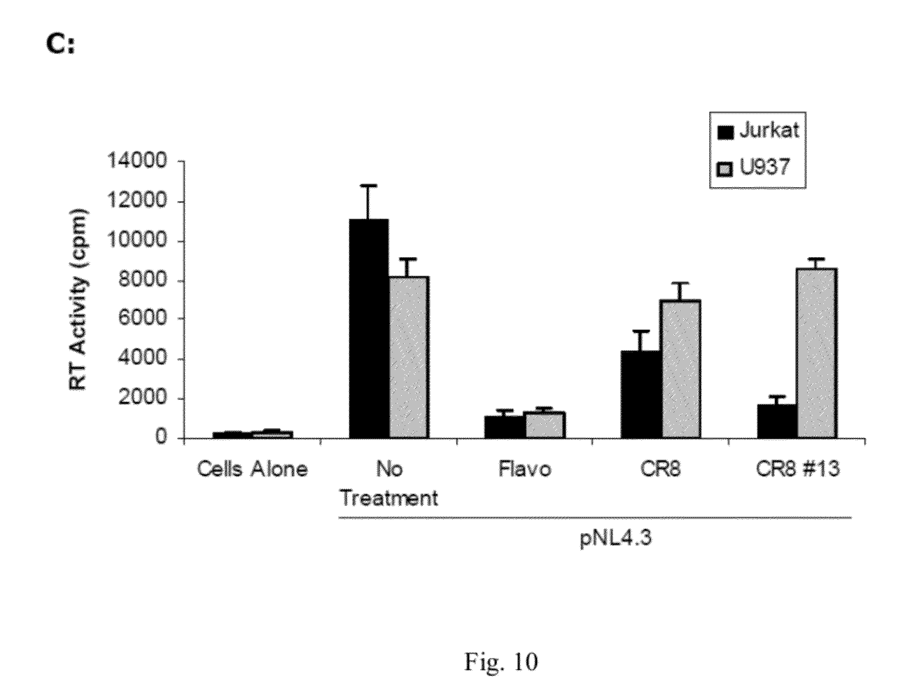

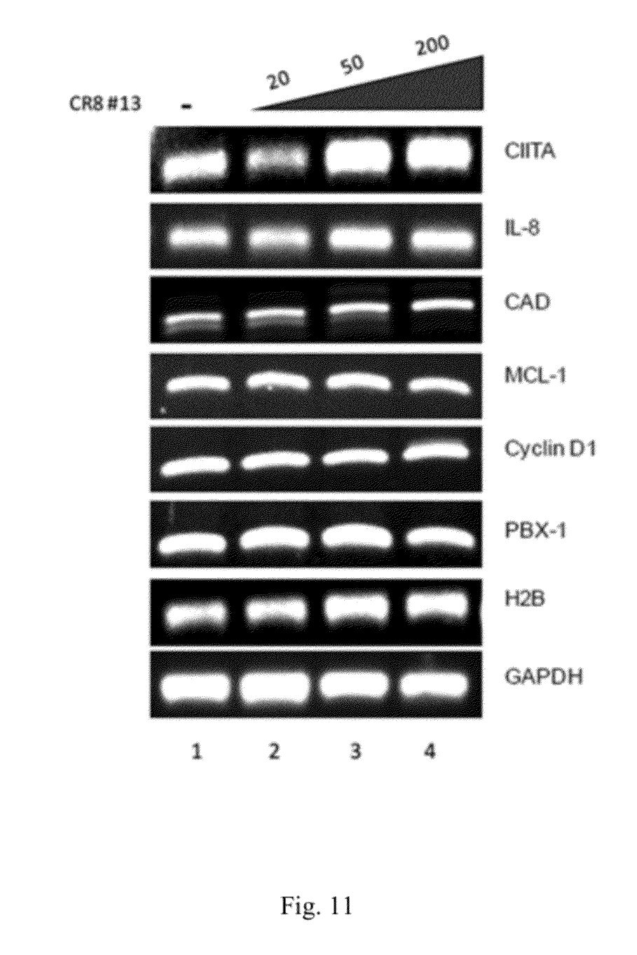

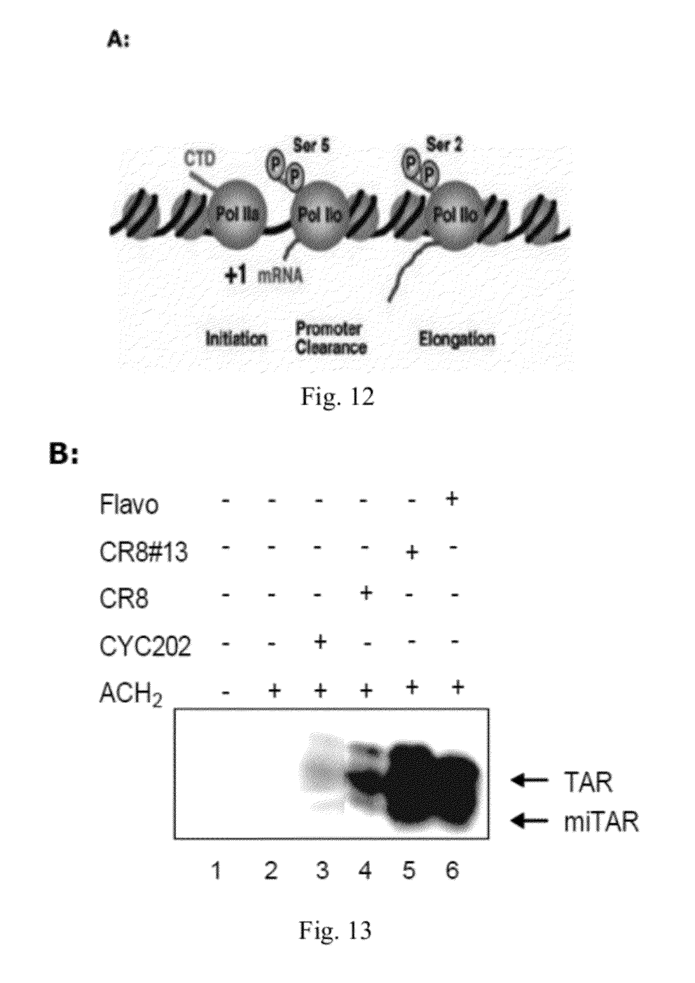

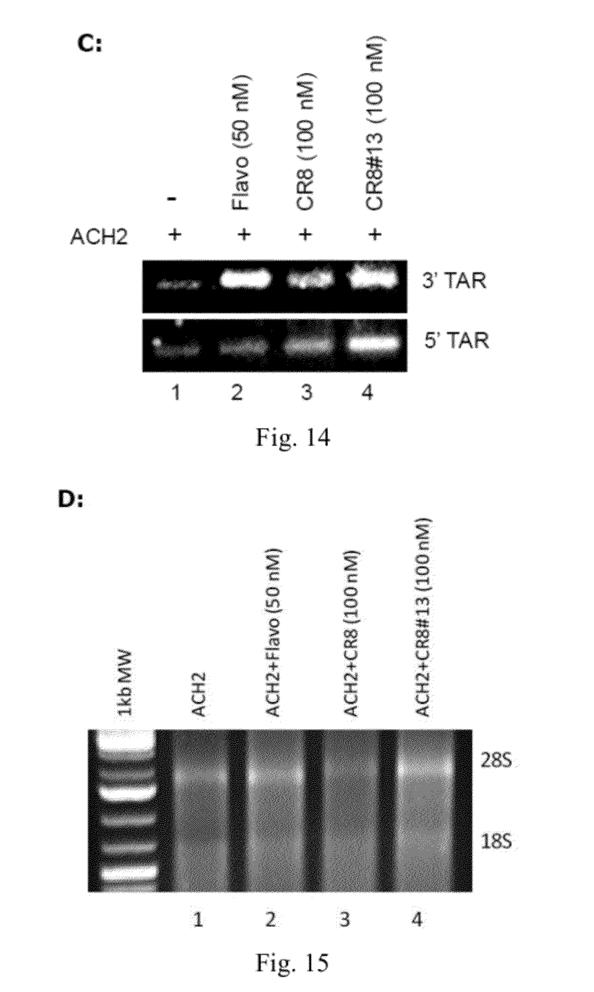

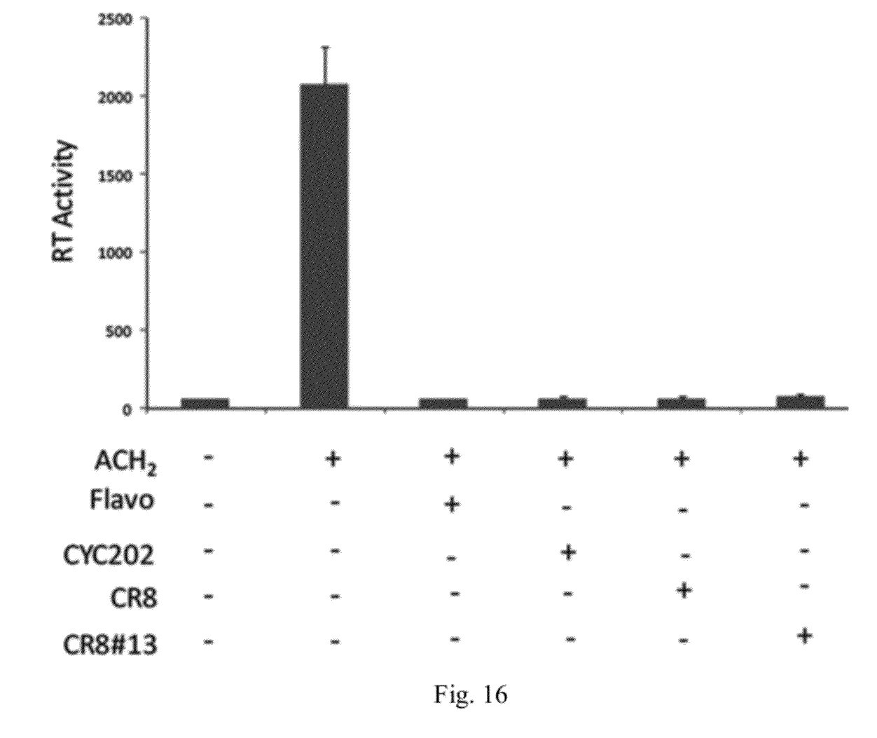

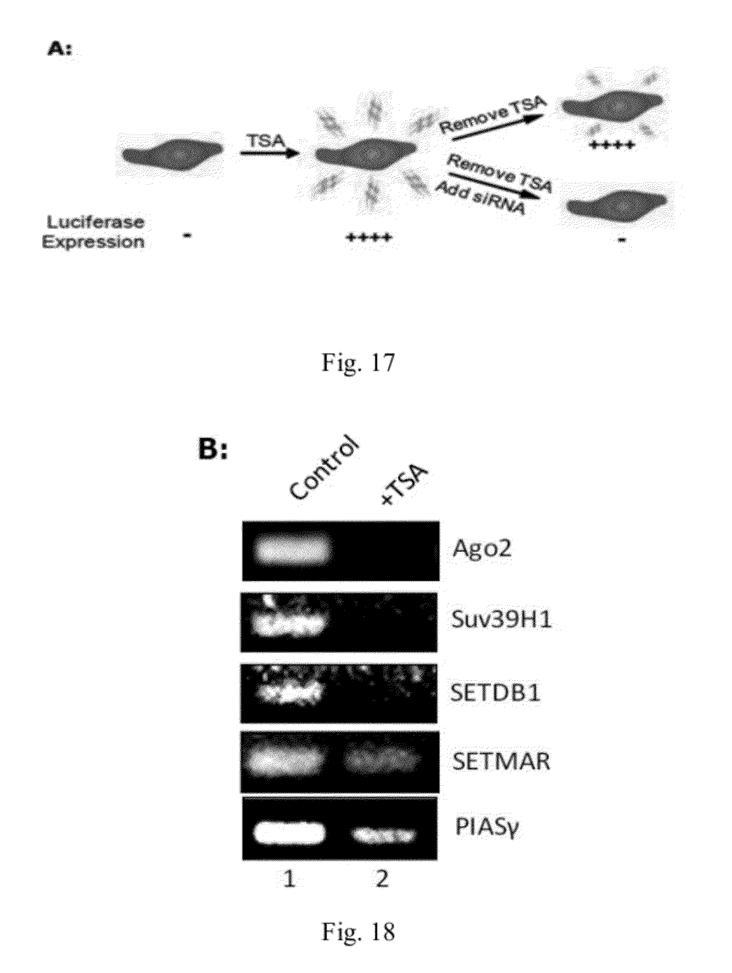

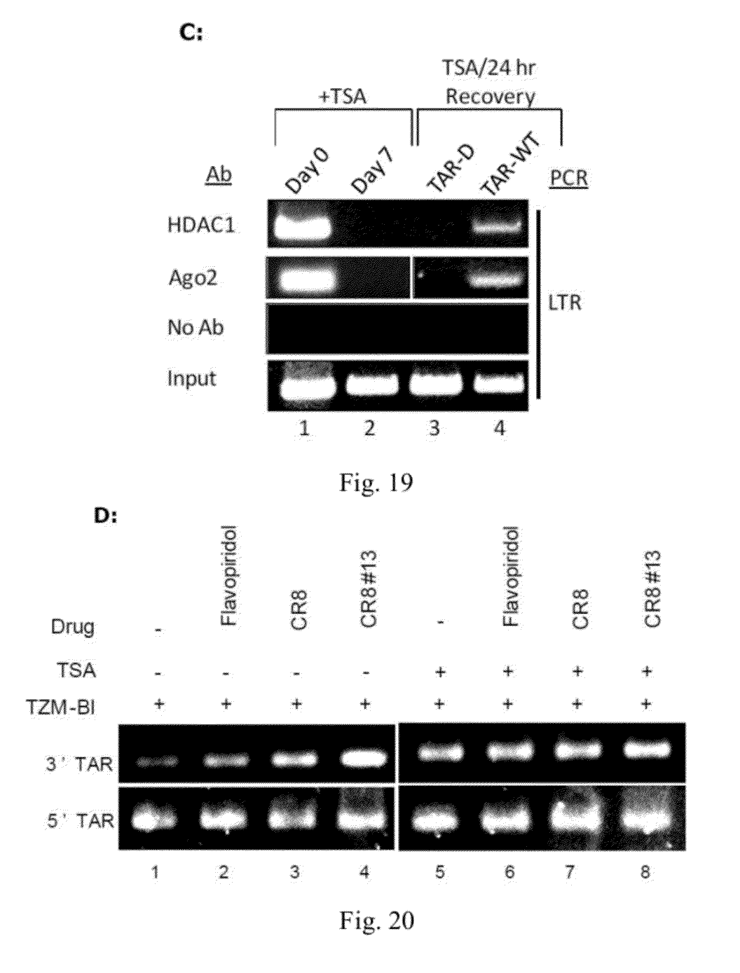

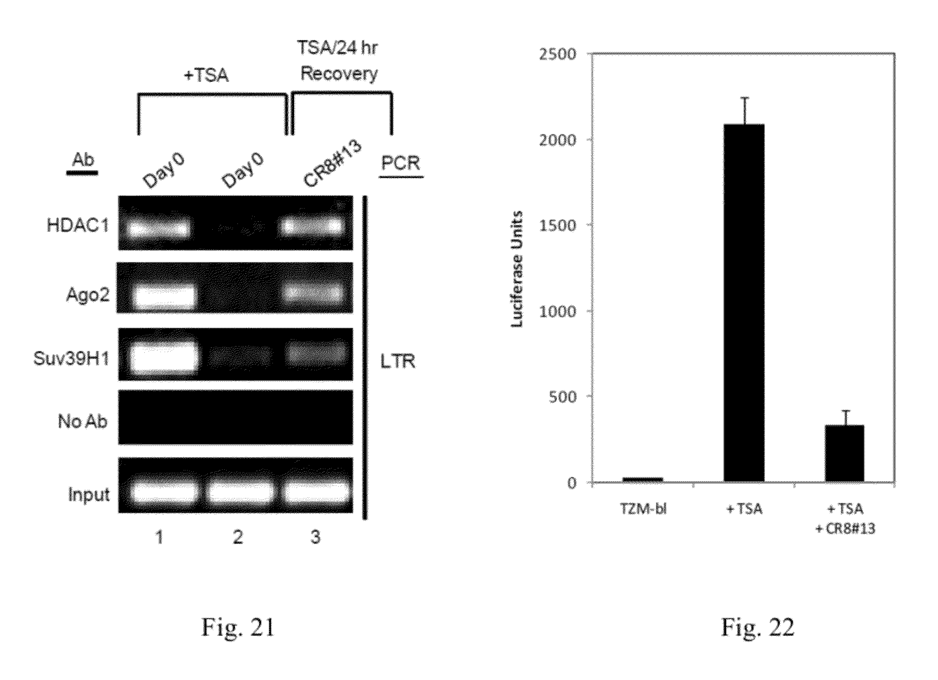

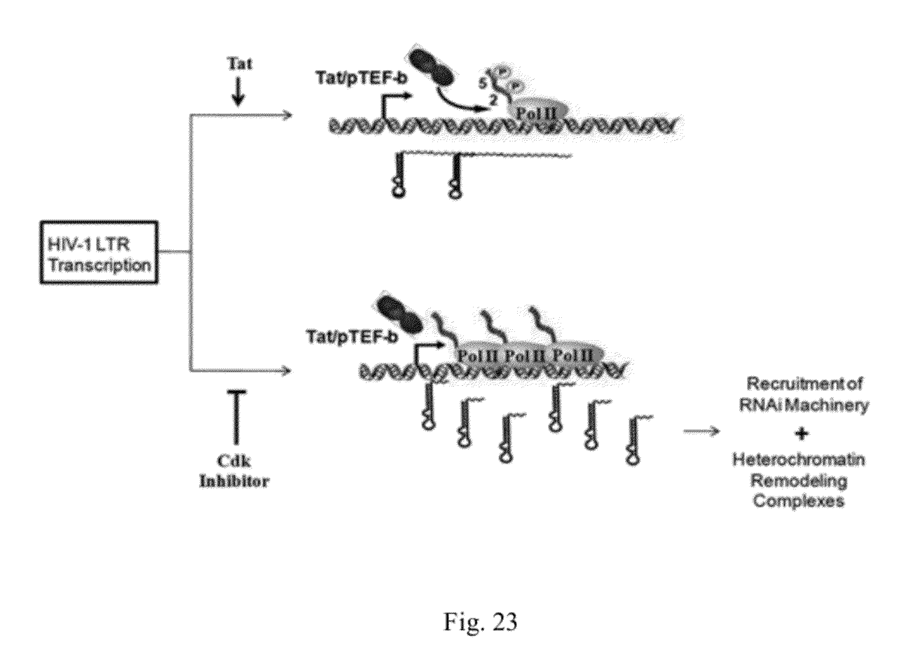

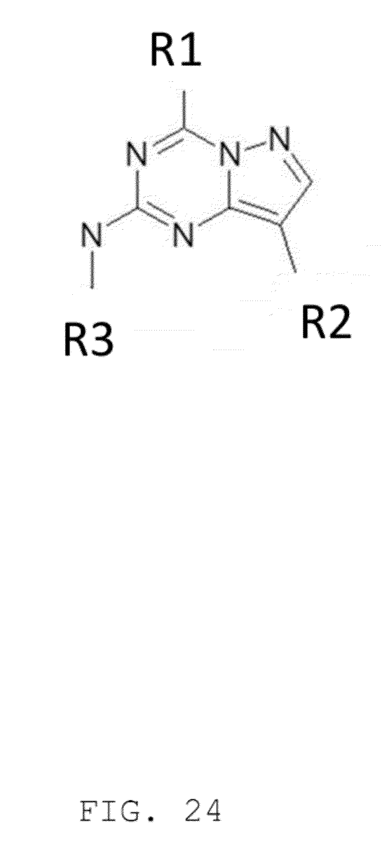

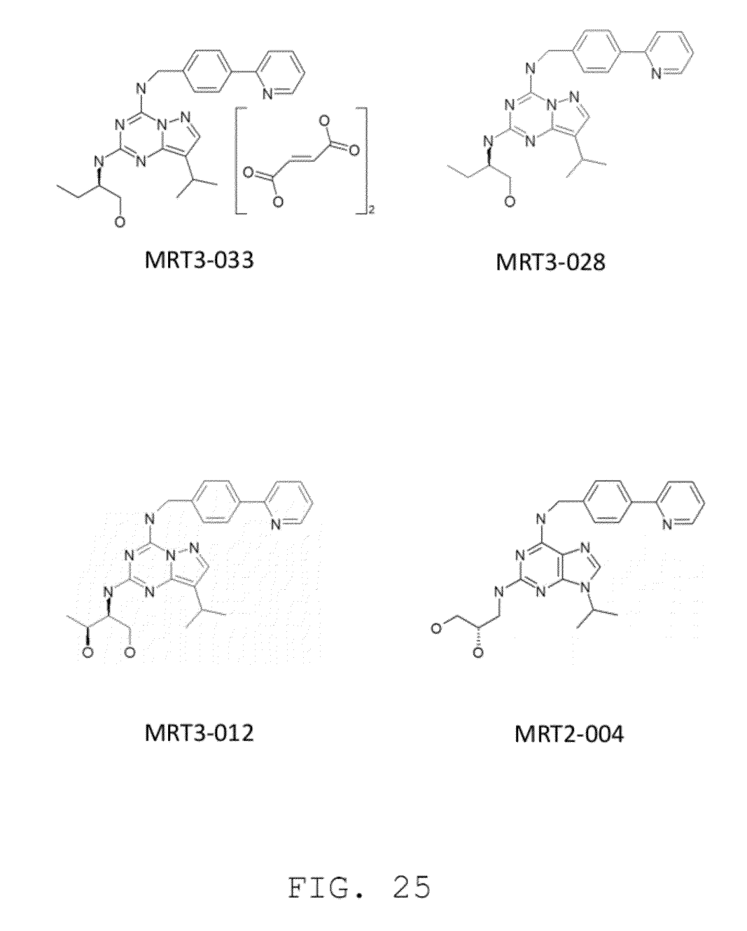

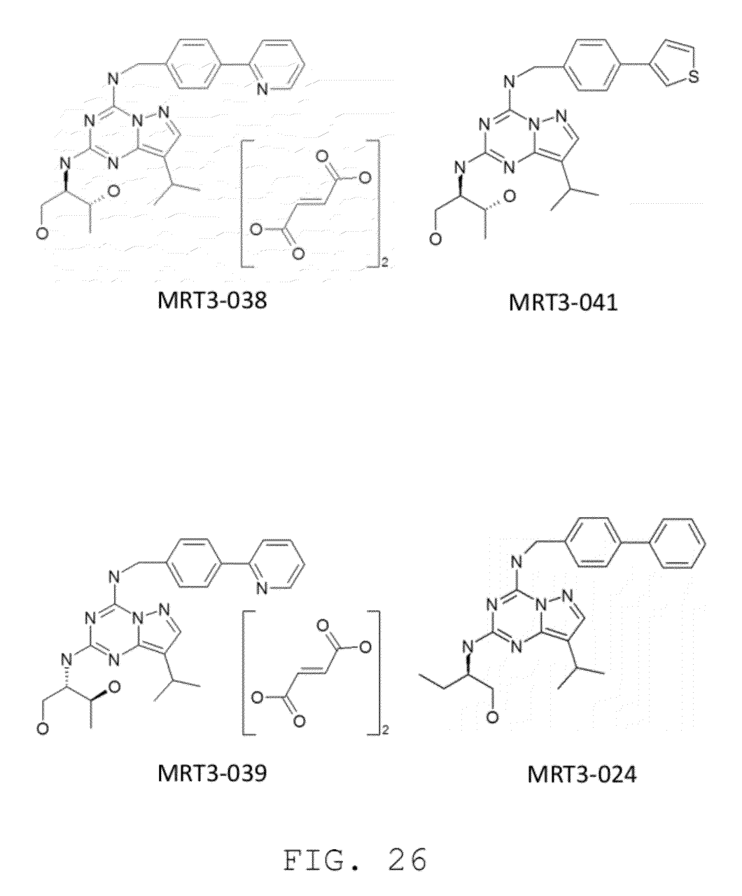

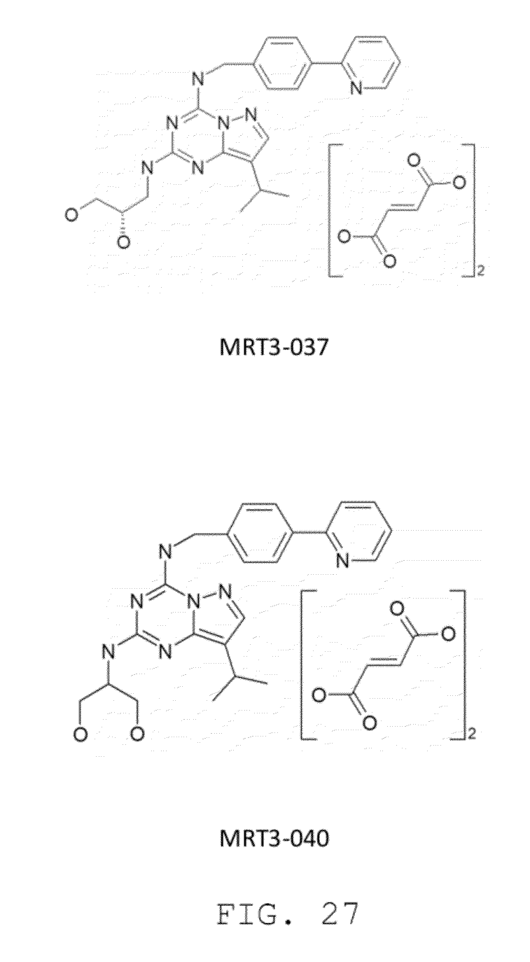

### DETAILED DESCRIPTION OF EMBODIMENTS

According to embodiments, a process to treat human immunodeficiency virus (HIV) infection includes administering to a subject a therapeutically effective amount of a viral transcription modulator shown below.

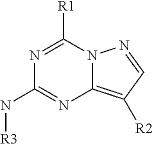

According to embodiments, viral transcription modulators include pharmaceutically acceptable salts or prodrug of the compound shown above. According to embodiments, at least one of R1, R2, and R3 includes a hydrocarbon.

According to further embodiments, said viral transcription modulator includes at least one of CR8#13(BJFP1154), MRT3-033, MRT3-028, MRT3-012, MRT3-038, MRT3-041, MRT3-039, MRT3-024, and MRT3-040.

In a further teaching, a viral transcription modulator includes CR8#13 (BJFP1154).

According to embodiments, a viral transcription modulator has an IC50 between approximately 1 nM to approximately 1000 nM. In a further embodiment, a viral transcription modulator has an IC50 below approximately 50 nM. In additional embodiments, a viral transcription modulator has an IC50 between approximately 10 nM.

According to embodiments, a subject includes at least one of a pediatric subject, a gravid subject, an adult subject and a lactating subject.

According to embodiments, a process to treat human immunodeficiency virus (HIV) infection may additionally comprise administering at least one of a nucleoside reverse transcriptase inhibitor, a nucleotide reverse transcriptase inhibitor, a non-nucleoside reverse transcriptase inhibitor, a protease inhibitor, an integrase inhibitor, an entry inhibitor, and a maturation inhibitor.

According to additional teachings, a method of modulating immunodeficiency viral LTR transcription in a cell may include contacting said cell with at least one of: CR8#13(BJFP1154), MRT3-0334, MRT3-028, MRT3-012, MRT3-038, MRT3-041, MRT3-039, MRT3-024, and MRT3-040, and a pharmaceutically acceptable salt and or prodrug thereof.

In embodiments, a modulator may include a viral transcription modulator.

According to embodiments, a modulator may include modulators which minimize or maximize viral transcription.

According to embodiments, viral transcription modulation includes modulation in a cell.

According to embodiments, a process may modulate viral transcription n a cell. According to additional embodiments, transcription may include LTR transcription.

According to embodiments, modulators include modulators which inhibit at least one of CDK 1, CDK 2, CDK 4, CDK 5, CDK 7, CDK 9, CDK 2/E, CDK 2/A, CD7, and CDK9.

In a further teaching, a process of treating HIV in a subject includes administering a compound having the structure of at least one of CR8#13 (BJFP1154), MRT3-0334, MRT3-028, MRT3-012, MRT3-038, MRT3-041, MRT3-039, MRT3-024, MRT3-040, and a pharmaceutically acceptable prodrug or salt thereof. In an additional teaching, a process of treating HIV includes a process of treating patients at risk for retroviral infection.

In a further teaching, a process of treating a patient at risk for retroviral infection includes administering a compound with the structure of at least one of CR8#13 (BJFP1154), MRT3-0334, MRT3-028, MRT3-012, MRT3-038, MRT3-041, MRT3-039, MRT3-024, MRT3-040, a pharmaceutically acceptable prodrug thereof, and a salt thereof.

In a further teaching, a process of reducing viral load comprises administering to a subject a therapeutically effective amount of at least one of CR8#13(BJFP1154), MRT3-033, MRT3-028, MRT3-012, MRT3-038, MRT3-041, MRT3-039, MRT3-024, MRT3-040, a pharmaceutically acceptable prodrug thereof, and a salt thereof wherein the viral load is reduced. An additional embodiment, the viral load includes the lytic viral load and latent viral load.

In an additional teaching, the use of at least one of CR8#13 (BJFP1154), MRT3-0334, MRT3-028, MRT3-012, MRT3-038, MRT3-041, MRT3-039, MRT3-024, MRT3-040, and a pharmaceutically acceptable salt or prodrug thereof, in the preparation of a medicament for the treatment of HIV infection is provided.

In an additional aspect of an embodiment, an article of manufacture comprising a vessel containing at least one of CR8#13 (BJFP1154), MRT3-0334, MRT3-028, MRT3-012, MRT3-038, MRT3-041, MRT3-039, MRT3-040, MRT3-024, MRT3-040, and a pharmaceutically acceptable prodrug or salt thereof, an instruction to treat an human immunodeficiency virus (HIV) infection in a subject. In another aspect of an embodiment, the instruction calls for administering at least one of said at least one of in an effective amount of at least one of said at least one of CR8#13 (BJFP1154), MRT3-0334, MRT3-028, MRT3-012, MRT3-038, MRT3-041, MRT3-039, MRT3-024, MRT3-040, and a pharmaceutically acceptable prodrug or salt thereof.

In an aspect of an embodiment, modulators, chemicals, and pharmaceutically acceptable prodrugs or salts include those that are administered in an effective amount.

In a further aspect of an embodiment, an article of manufacture is provided. In a further aspect of an embodiment, an article of manufacture includes a vessel wherein a vessel comprises at least one of a nucleoside reverse transcriptase inhibitor, a nucleotide reverse transcriptase inhibitor, a non-nucleoside reverse transcriptase inhibitor, a protease inhibitor, an integrase inhibitor, an entry inhibitor, and a maturation inhibitor; and said instruction comprises administering at least of said at least one of a nucleoside reverse transcriptase inhibitor, a nucleotide reverse transcriptase inhibitor, a non-nucleoside reverse transcriptase inhibitor, a protease inhibitor, an integrase inhibitor, an entry inhibitor, and a maturation inhibitor.

In an aspect of an embodiment, an article of manufacture may comprise packaging material and contained within the packaging material at least one of CR8#13 (BJFP1154), MRT3-0334, MRT3-028, MRT3-012, MRT3-038, MRT3-041, MRT3-039, MRT3-024, MRT3-040, and a pharmaceutically acceptable salt and/or prodrug thereof. In a further aspect of an embodiment, a vessel may include a vessel containing at least one antiretroviral drug. In a further teaching, packaging material may comprise a label that indicates that said at least one of CR8#13 (BJFP1154), MRT3-0334, MRT3-028, MRT3-012, MRT3-038, MRT3-041, MRT3-039, MRT3-024, MRT3-040, and/or a pharmaceutically acceptable prodrug or salt thereof may be used for treating HIV infection.

In embodiments, at least one of R1, R2, and R3 may include groups other than hydrocarbons.

In other embodiments, the subject includes a subject with at least one white blood cell.

According to embodiments, compositions may be administered orally, parenterally, transdermally, bucally, nasally, mucosally, and sublingually or any combination thereof. According to embodiments, antiretroviral drugs include drugs that target various phases of the retrovirus-life-cycle. Antiretroviral drugs include nucleoside and nucleotide reverse transcriptase inhibitors (NRTI), non-nucleoside reverse transcriptase inhibitors, protease inhibitors, integrase inhibitors, entry inhibitors, and maturation inhibitors. Antiretroviral drugs include emtricitabine (an NRTI), tenofovir (an NRTI), efavirenz (a NNRTI), raltegravir (an integrase inhibitor), darunavir (a protease inhibitor), ritonavir (a protease inhibitor), atazanavir (a protease inhibitor), zidovudine, lamivudine, abacavir, lopinavir, stavudine, lamivudine, and nevirapine.

According to embodiments, antiretroviral drugs include combivir (Glaxo Smith Kline), emtriva (Gilead sciences), epivir (GlaxoSmithKline), Epivir (Glaxo Smith Klein), Epzicom (Glaxo Smith Kline), Hivid (Hoffman-La Roche), Retrovir, Trizivir (Glaxo Smith Kline), lamivudine and zidovudine (GlaxoSmithKline), Emtricitabine (Gilead Sciences), lamivudine, 3TC (GlaxoSmithKline), abacavir/lamivudine (GlaxoSmithKline), zalcitabine, ddC, dideoxycytidine (Hoffmann-La Roche), zidovudine, AZT, azidothymidine, ZDV (Glaxo Smith Kline), abacavir, zidovudine, and lamivudine (Glaxo Smith Kline), tenofovir disoproxil/emtricitabine (Gilead Sciences, Inc.), enteric coated didanosine (Bristol Myers-Squibb), didanosine, ddI, dideoxyinosine (Bristol Myers-Squibb), tenofovir disoproxil fumarate (Gilead Sciences), stavudine, d4T (Bristol Myers-Squibb), and abacavir (Glaxo Smith Kline).

According to embodiments, nonnucleoside reverse transcriptase inhibitors (NNRTIs) include delavirdine, DLV (Pfizer), efavirenz (Bristol Myers-Squibb), nevirapine (BI-RG-587, Boehringer Ingelheim).

According to embodiments, protease Inhibitors (PIs) include amprenavir (GlaxoSmithKline), Tipranavir (Boehringer Ingelheim), saquinavir mesylate, SQV (Hoffmann-La Roche), lopinavir and ritonaviry (Abbott Laboratories), Fosamprenavir Calcium (GlaxoSmithKline), ritonavir, ABT-538 (Abbott Laboratories), darunavir (Tibotec, Inc.), atazanavir sulfate (Bristol-Myers Squibb), and nelfinavir mesylate, NFV(Agouron Pharmaceuticals).

According to embodiments, fusion inhibitors include enfuvirtide, T-20 (Hoffmann-La Roche & Trimeris).

In embodiments, entry inhibitors include maraviroc (Pfizer).

According to embodiments, HIV integrase strand-transfer inhibitors include raltegravir (Merck & Co., Inc.).

According to embodiments anti-HIV treatment regimens may include combinations of drugs. According to embodiments multiclass combinations of drugs may include combivir (lamivudine and zidovudine, GlaxoSmithKline), emtriva (Emtricitabine and FTC, Gilead Sciences), epivir (lamivudine and 3TC, GlaxoSmithKline), epzicom (abacavir and lamivudine, GlaxoSmithKline), hivid (zalcitabine, dideoxycytidine, ddC (Hoffmann-La Roche), Retrovir (zidovudine, azidothymidine, AZT, ZDV (GlaxoSmithKline), Trizivir (abacavir, zidovudine, and lamivudine (GlaxoSmithKline), Truvada (tenofovir disoproxil fumarate and emtricitabine, Gilead Sciences, Inc.), videx EC (enteric coated didanosine, ddI EC, Bristol Myers-Squibb), videx (didanosine, dideoxyinosine, ddI, Bristol Myers-Squibb), viread (tenofovir disoproxil fumarate, TDF, Gilead), zerit (stavudine, d4T, Bristol Myers-Squibb), ziagen (abacavir sulfate, ABC (GlaxoSmithKline).

In this specification, “a” and “an” and similar phrases are to be interpreted as “at least one” and “one or more.” References to “an” embodiment in this disclosure are not necessarily to the same embodiment.

The disclosure of this patent document incorporates material which is subject to copyright protection. The copyright owner has no objection to the facsimile reproduction by anyone of the patent document or the patent disclosure, as it appears in the Patent and Trademark Office patent file or records, for the limited purposes required by law, but otherwise reserves all copyright rights whatsoever.

While various embodiments have been described above, it should be understood that they have been presented by way of example, and not limitation. It will be apparent to persons skilled in the relevant art(s) that various changes in form and detail can be made therein without departing from the spirit and scope. In fact, after reading the above description, it will be apparent to one skilled in the relevant art(s) how to implement alternative embodiments. Thus, the present embodiments should not be limited by any of the above described exemplary embodiments.

In addition, it should be understood that any figures that highlight any functionality and/or advantages, are presented for example purposes only.

Further, the purpose of the Abstract of the Disclosure is to enable the U.S. Patent and Trademark Office and the public generally, and especially the scientists, engineers and practitioners in the art who are not familiar with patent or legal terms or phraseology, to determine quickly from a cursory inspection the nature and essence of the technical disclosure of the application. The Abstract of the Disclosure is not intended to be limiting as to the scope in any way.

Finally, it is the applicant's intent that only claims that include the express language “means for” or “step for” be interpreted under 35 U.S.C. 112, paragraph 6.

Claims that do not expressly include the phrase “means for” or “step for” are not to be interpreted under 35 U.S.C. 112, paragraph 6.

What is claimed is:

1. A process to treat human immunodeficiency virus (HIV) infection comprising administering to a subject a therapeutically effective amount of a viral transcription modulator, a pharmaceutically acceptable salt thereof or prodrug thereof, the viral transcription modulator comprising the structure: 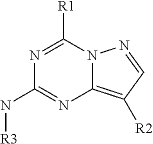
wherein at least one of R1, R2, and R3 is a hydrocarbon.

2. The process of claim 1, wherein said viral transcription modulator includes at least one of CR8#13 (BJFP1154), MRT3-033, MRT3-028, MRT3-012, MRT3-038, MRT3-041, MRT3-039, MRT3-024, and MRT3-040.

3. The process of claim 1, wherein said viral transcription modulator includes CR8#13 (BJFP1154).

4. The process of claim 0, wherein said viral transcription modulator has an IC50 between approximately 1 nM to approximately 1000 nM.

5. The process of claim 0, wherein said viral transcription modulator has an IC50 below approximately 50 nM.

6. The process of claim 5, wherein said viral transcription modulator has an IC50 between approximately 10 nM.

7. The process of claim 1, wherein said subject includes at least one of a pediatric subject, a gravid subject, an adult subject and a lactating subject.

8. The process of claim 1, further comprising administering at least one of a nucleoside reverse transcriptase inhibitor, a nucleotide reverse transcriptase inhibitor, a non-nucleoside reverse transcriptase inhibitor, a protease inhibitor, an integrase inhibitor, an entry inhibitor, and a maturation inhibitor.
